(19) United States
(12) Patent Application Publication
(10) Pub. No.: US 2007/0276048 A1
(43) Pub. Date: Nov. 29, 2007

(54) UNIT DOSE FORMULATIONS COMPRISING AN INHALABLE SOLUTION OF ALBUTEROL

(75) Inventors: Hemant Deshmukh, San Diego, CA (US); Elaine Phillips, San Diego, CA (US); Malcolm Hill, Solana Beach, CA (US)

Correspondence Address: WILSON SONSINI GOODRICH & ROSATI, 650 PAGE MILL ROAD, PALO ALTO, CA, 94304-1050, US

(73) Assignee: Verus Pharmaceuticals, Inc., San Diego, CA (US)

(21) Appl. No.: 11/750,992

(22) Filed: May 18, 2007

Related U.S. Application Data
(60) Provisional application No. 60/747,657, filed on May 18, 2006.
(60) Provisional application No. 60/803,232, filed on May 25, 2006.
(60) Provisional application No. 60/828,212, filed on Oct. 4, 2006.
(60) Provisional application No. 60/828,215, filed on Oct. 4, 2006.

(51) Int. Cl. A61K 31/138

(52) U.S. Cl. 514/651

(57) ABSTRACT

The present invention relates to unit dose formulations comprising an inhalable albuterol solution, wherein the inhalation albuterol solution is formulated with albuterol free base, a tonicity adjusting agent, a pH adjusting agent and water. Methods of preparing the unit dose formulation are also provided.

### RELATED APPLICATIONS

This application claims the benefit of U.S. Provisional Application Nos. 60/747,657, filed May 18, 2006; 60/803,232, filed May 25, 2006; 60/828,212, filed Oct. 4, 2006; and 60/828,215, filed Oct. 4, 2006, which are hereby incorporated by reference in their entireties.

### FIELD OF THE INVENTION

The present invention relates to unit dose formulations comprising an inhalable albuterol solution, wherein the inhalation albuterol solution is formulated with albuterol free base, a tonicity adjusting agent, a pH adjusting agent and water. Methods of preparing the unit dose formulation are also provided.

### BACKGROUND OF THE INVENTION

Bronchoconstrictive disorders can include such pulmonary diseases as asthma and its related disorders, including pediatric asthma, bronchial asthma, allergic asthma, occupational asthma, aspirin sensitive asthma, intrinsic asthma, and chronic obstructive pulmonary disease (COPD), and chronic bronchitis. Such bronchoconstrictive disorders are widespread and affect millions of people worldwide.

The pathophysiology of many bronchoconstrictive disorders, including, asthma, involves various distinct symptoms, one of which is bronchoconstriction, which can result in wheezing, coughing and shortness of breath. It is believed that in these disorders, bronchoconstriction occurs due to one or more of bronchial smooth muscle spasms, airway inflammation and bronchial mucosal edema.

In particular, asthma is a bronchoconstrictive disorder marked by (a) labored breathing; (b) wheezing; and (c) coughing. Like many bronchoconstrictive disorders, asthma is characterized by: (1) airway inflammation; (2) airway hyper-responsiveness; and (3) airway narrowing. However, the severity of these symptoms can vary widely from patient to patient and even from one asthmatic episode (attack) to the next within the same patient.

β₂ agonists, also known in the art as β₂-adrenergic receptor agonists, are known to provide a bronchodilatory effect in humans and are important in the treatment of patients suffering from bronchoconstrictive disorders because the administration of β₂ agonists results in relief from the symptoms of breathlessness. The β₂ agonists can be short acting for immediate relief, or long acting for long-term prevention, of bronchoconstrictive symptoms. For example, known short acting β₂ agonists include albuterol, biltolterol, levalbuterol, pirbuterol, salbutamol, or terbutaline. Additionally, known long acting β₂ agonists include arformoterol, formoterol and salmeterol.

Short-acting inhaled β₂ agonists, such as albuterol, are used to prevent and treat wheezing, shortness of breath, and troubled breathing caused by asthma, chronic bronchitis, emphysema, and other lung diseases. β₂ agonist inhalation is also used to prevent breathing difficulties (bronchospasm) during exercise. Currently, albuterol is available as a tablet, extended-release (long-acting) tablet, and a syrup to take by mouth and as an aerosol, a solution (liquid), and a powder-filled capsule to inhale by mouth. The solution is inhaled using a nebulizer, and the powder-filled capsules are inhaled using a special dry powder inhaler. Albuterol tablets and syrup are usually taken three or four times a day, and extended-release tablets are usually taken twice a day. For the treatment or prevention of asthma symptoms, the oral inhalation is usually used every 4 to 6 hours as needed. For the prevention of bronchospasm during exercise, the oral inhalation is used 15 minutes before exercise. The nebulized solution is used three or four times a day.

An inhalation solution of albuterol is currently available in 2.5 mg, 1.25 mg, and 0.63 mg unit doses in 3 mls of an isotonic aqueous solution (Albuterol Sulfate Inhalation Solution and Accuneb®, respectively (Dey, L. P.). The 2.5 mg dose has been approved for use by adults, and the FDA has likewise expanded labeling guidelines to include this amount of albuterol sulfate for use by pediatric asthmatic patients as young as 2 years old. Although, when administered on a regular basis to a child, the 2.5 mg albuterol sulfate formulation may provide more albuterol than needed, and thereby increase the risk of adverse drug side effects. As such, the National Institutes of Health (NIH) has recommended that pediatric patients use the lowest β2 agonist dose needed to control symptoms.

In addition, it is known that albuterol undergoes degradation in aqueous solutions to form albuterol aldehyde. Albuterol aldehyde has potential negative effects when administered by inhalation and therefore its level in inhalation solutions is controlled by the U.S. Food and Drug Administration. The rate of degradation of albuterol in aqueous solutions, to albuterol aldehyde, increases with increasing initial drug concentration (Malkki et al. (1990) Int. J. Pharmaceutics 63:17-22).

Therefore, there is a need to provide stabilized albuterol compositions. One approach to reduce the levels of albuterol aldehyde described earlier has been to blow nitrogen gas over the solution during formulation and filling of the solution in unit dose vials. The vials are then enclosed in an oxygen-impermeable wrapper in a reduced oxygen atmosphere (U.S. Pat. No. 6,451,289). This process is cumbersome and it is difficult to control the level of oxygen during manufacturing and storage. Once the protective wrapper is opened, albuterol is exposed to ambient oxygen and degradation can occur.

Accordingly, there is a need for improved unit dosage formulations comprising β₂ agonists for use in the treatment of bronchoconstrictive disorders in a patient in need thereof, wherein the β₂ agonists are delivered in a manner in which β₂ agonist unit dosage volume is lowered and wherein the risk of side effects related to β₂ agonist therapy is diminished.

The present invention meets the foregoing needs and provides related advantages as well.

### SUMMARY OF THE INVENTION

The present invention meets the foregoing and related needs by providing an improved method of treating bronchoconstrictive disorders, including asthma, with β₂ agonists where current treatments are not ideal.

In certain embodiments of the present invention, the invention comprises a unit dose formulation comprising a therapeutically effective amount of an albuterol solution prepared by the process comprising the steps of: (a) providing about 0.84 to about 0.90 weight % NaCl; (b) mixing said NaCl with about 80 weight % water to form an aqueous solution; (c) adding from about 0.03 to about 0.25 weight % albuterol free base to the solution of (b); (d) adding about 0.10 to about 1.3 weight % HCl (1N) to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: HCl of about 0.85 to about 1.20, a pH of about 3.0 to about 4.5, and the unit dose formulation comprises a therapeutically effective amount of albuterol and is suitable for inhalation therapy via nebulization. In other embodiments, the process can optionally further comprise the addition of NaOH in an amount sufficient to increase the pH of the unit dose formulation to about 3.0 to about 4.5, if necessary.

In one embodiment of the present invention, the invention comprises a unit dose formulation comprising a therapeutically effective amount of an albuterol solution prepared by the process comprising the steps of: (a) providing about 0.90 weight % NaCl; (b) mixing said NaCl with about 80 weight % water to form an aqueous solution; (c) adding about 0.03 weight % albuterol free base to the solution of (b); (d) adding about 0.17 weight % HCl (1N) to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: HCl of about 0.87, a pH of about 3.7, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary.

In another embodiment of the present invention, the invention comprises a unit dose formulation comprising a therapeutically effective amount of an albuterol solution prepared by the process comprising the steps of: (a) providing about 0.87 weight % NaCl; (b) mixing said NaCl with about 80 weight % water to form an aqueous solution; (c) adding about 0.12 weight % albuterol free base to the solution of (b); (d) adding about 0.55 weight % HCl (1N) to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: HCl is about 1.08, a pH of about 3.6, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.6, if necessary.

In yet another embodiment of the present invention, the invention comprises a unit dose formulation comprising a therapeutically effective amount of an albuterol solution prepared by the process comprising the steps of: (a) providing about 0.84 weight % NaCl; (b) mixing said NaCl with about 80 weight % water to form an aqueous solution; (c) adding about 0.25 weight % albuterol free base to the solution of (b); (d) adding about 1.06 weight % HCl (1N) to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: HCl of about 1.16, a pH of about 3.4, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.4, if necessary.

In yet another embodiment of the present invention, the invention comprises a unit dose formulation comprising a therapeutically effective amount of an albuterol solution prepared by the process comprising the steps of: (a) providing about 0.88 weight % NaCl; (b) mixing said NaCl with about 80 weight % water to form an aqueous solution; (c) adding about 0.062 weight % albuterol free base to the solution of (b); (d) adding about 0.33 weight % HCl (1N) to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: HCl of about 0.94, a pH of about 3.75±0.15, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.75±0.15, if necessary.

In yet another embodiment of the present invention, the invention comprises a unit dose formulation comprising a therapeutically effective amount of an albuterol solution prepared by the process comprising the steps of: (a) providing about 0.86 weight % NaCl; (b) mixing said NaCl with about 80 weight % water to form an aqueous solution; (c) adding about 0.126 weight % albuterol free base to the solution of (b); (d) adding about 0.64 weight % HCl (1N) to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: HCl of about 0.97, a pH of about 3.75±0.15, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.75±0.15, if necessary.

In yet another embodiment of the present invention, the invention comprises a unit dose formulation comprising a therapeutically effective amount of an albuterol solution prepared by the process comprising the steps of: (a) providing about 0.84 weight % NaCl; (b) mixing said NaCl with about 80 weight % water to form an aqueous solution; (c) adding about 0.25 weight % albuterol free base to the solution of (b); (d) adding about 1.30 weight % HCl (1N) to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: HCl of about 0.95, a pH of about 3.75±0.15, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.75±0.15, if necessary.

In certain embodiments of the present invention, the invention comprises a unit dose formulation comprising a therapeutically effective amount of an albuterol solution prepared by the process comprising the steps of: (a) providing about 0.84 to about 0.90 weight % NaCl; (b) mixing said NaCl with about 80 weight % water to form an aqueous solution; (c) adding from about 0.03 to about 0.25 weight % albuterol free base to the solution of (b); (d) adding about 0.03 to about 0.35 weight % citric acid to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: citric acid of about 0.80 to about 1.0, a pH of about 3.0 to about 4.5, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.0 to about 4.5, if necessary. In an alternate embodiment, the process can optionally further comprise the addition of sodium citrate in an amount sufficient to increase the pH of the unit dose formulation to about 3.0 to about 4.5, if necessary.

In one embodiment of the present invention, the invention comprises a unit dose formulation comprising a therapeutically effective amount of an albuterol solution prepared by the process comprising the steps of: (a) providing about 0.90 weight % NaCl; (b) mixing said NaCl with about 80 weight % water to form an aqueous solution; (c) adding about 0.03 weight % albuterol free base to the solution of (b); (d) adding about 0.03 weight % citric acid to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: citric acid is about 0.80, a pH of about 3.7, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium citrate in an amount sufficient to increase the pH of the unit dose formulation to about 3.7, if necessary.

In another embodiment of the present invention, the invention comprises a unit dose formulation comprising a therapeutically effective amount of an albuterol solution prepared by the process comprising the steps of: (a) providing about 0.87 weight % NaCl; (b) mixing said NaCl with about 80 weight % water to form an aqueous solution; (c) adding about 0.12 weight % albuterol free base to the solution of (b); (d) adding about 0.11 weight % citric acid to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: citric acid of about 0.88, a pH of about 3.7, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium citrate in an amount sufficient to increase the pH of the unit dose formulation to about 3.7, if necessary.

In yet another embodiment of the present invention, the invention comprises a unit dose formulation comprising a therapeutically effective amount of an albuterol solution prepared by the process comprising the steps of: (a) providing about 0.84 weight % NaCl; (b) mixing said NaCl with about 80 weight % water to form an aqueous solution; (c) adding about 0.25 weight % albuterol free base to the solution of (b); (d) adding about 0.21 weight % citric acid to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: citric acid of about 0.96, a pH of about 3.7, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium citrate in an amount sufficient to increase the pH of the unit dose formulation to about 3.7, if necessary.

In certain embodiments of the present invention, the invention comprises a unit dose formulation comprising a therapeutically effective amount of an albuterol solution prepared by the process comprising the steps of: (a) providing about 0.84 to about 0.90 weight % NaCl; (b) mixing said NaCl with about 80 weight % water to form an aqueous solution; (c) adding from about 0.03 to about 0.25 weight % albuterol free base to the solution of (b); (d) adding about 0.10 to about 1.20 weight % H₃PO₄ to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: H₃PO₄ of about 0.80 to about 1.20, a pH of about 3.0 to about 4.5, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.0 to about 4.5, if necessary. In an alternate embodiment, the process can optionally further comprise the addition of sodium phosphate dibasic in an amount sufficient to increase the pH of the unit dose formulation to about 3.0 to about 4.5, if necessary.

In one embodiment of the present invention, the invention comprises a unit dose formulation comprising a therapeutically effective amount of an albuterol solution prepared by the process comprising the steps of; (a) providing about 0.90 weight % NaCl; (b) mixing said NaCl with about 80 weight % water to form an aqueous solution; (c) adding about 0.03 weight % albuterol free base to the solution of (b); (d) adding about 0.10 weight % H₃PO₄ to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: H₃PO₄ of about 0.80 to about 1.20, a pH of about 3.7, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium phosphate dibasic in an amount sufficient to increase the pH of the unit dose formulation to about 3.7, if necessary.

In another embodiment of the present invention, the invention comprises a unit dose formulation comprising a therapeutically effective amount of an albuterol solution prepared by the process comprising the steps of: (a) providing about 0.87 weight % NaCl; (b) mixing said NaCl with about 80 weight % water to form an aqueous solution; (c) adding about 0.12 weight % albuterol free base to the solution of (b); (d) adding about 0.34 weight % H₃PO₄ to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: H₃PO₄ of about 0.80 to about 1.20, a pH of about 3.7, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium phosphate dibasic in an amount sufficient to increase the pH of the unit dose formulation to about 3.7, if necessary.

In yet another embodiment of the present invention, the invention comprises a unit dose formulation comprising a therapeutically effective amount of an albuterol solution prepared by the process comprising the steps of: (a) providing about 0.84 weight % NaCl; (b) mixing said NaCl with about 80 weight % water to form an aqueous solution; (c) adding about 0.25 weight % albuterol free base to the solution of (b); (d) adding about 1.02 weight % H₃PO₄ to the solution of (c); (e) adding water to the solution of (d) in a quantity sufficient to provide a total weight % equal to 100; (f) filter sterilizing the solution of (e); and (g) dividing the solution of (f) into about 0.5 ml unit doses; wherein the unit dose formulation has a molar ratio of starting albuterol free base: H₃PO₄ of about 0.80 to about 1.20, a pH of about 3.7, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium phosphate dibasic in an amount sufficient to increase the pH of the unit dose formulation to about 3.7, if necessary.

In other embodiments of the present invention, the invention comprises a unit dose formulation comprising a therapeutically effective amount of an albuterol solution prepared by the process comprising the steps of: (a) providing about 0.84 to about 0.90 weight % NaCl; (b) mixing said NaCl with about 80 weight % water to form an aqueous solution; (c) adding from about 0.03 to about 0.25 weight % albuterol free base to the solution of (b); (d) adding about 0.001 to about 5.0 weight % of a stabilizing agent to the solution of (c); (e) adding about 0.10 to about 1.3 weight % HCl (1N) to the solution of (d); (f) adding water to the solution of (e) in a quantity sufficient to provide a total weight % equal to 100; (g) filter sterilizing the solution of (f); and (h) dividing the solution of (g) into about 0.5 ml unit doses; wherein the unit dose formulation has a molar ratio of starting albuterol free base: HCl of about 0.85 to about 1.20, a pH of about 3.0 to about 4.5, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.0 to about 4.5, if necessary.

In still other embodiments of the present invention, the invention comprises a unit dose formulation comprising a therapeutically effective amount of an albuterol solution prepared by the process comprising the steps of: (a) providing about 0.84 to about 0.90 weight % NaCl; (b) mixing said NaCl with about 80 weight % water to form an aqueous solution; (c) adding from about 0.03 to about 0.25 weight % albuterol free base to the solution of (b); (d) adding about 0.001 to about 5.0 weight % of a stabilizing agent to the solution of (c); (e) adding about 0.03 to about 0.35 weight % citric acid to the solution of (d); (f) adding water to the solution of (e) in a quantity sufficient to provide a total weight % equal to 100; (g) filter sterilizing the solution of (f); and (h) dividing the solution of (g) into about 0.5 ml unit doses; wherein the unit dose formulation has a molar ratio of starting albuterol free base: citric of about 0.80 to about 1.0, a pH of about 3.0 to about 4.5, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.0 to about 4.5, if necessary. In an alternate embodiment, the process can optionally further comprise the addition of sodium citrate in an amount sufficient to increase the pH of the unit dose formulation to about 3.0 to about 4.5, if necessary.

In yet other embodiments of the present invention, the invention comprises a unit dose formulation comprising a therapeutically effective amount of an albuterol solution prepared by the process comprising the steps of: (a) providing about 0.84 to about 0.90 weight % NaCl; (b) mixing said NaCl with about 80 weight % water to form an aqueous solution; (c) adding from about 0.03 to about 0.25 weight % albuterol free base to the solution of (b); (d) adding about 0.001 to about 5.0 weight % of a stabilizing agent to the solution of (c); (e) adding about 0.10 to about 1.20 weight % H₃PO₄ to the solution of (d); (f) adding water to the solution of (e) in a quantity sufficient to provide a total weight % equal to 100; (g) filter sterilizing the solution of (f); and (h) dividing the solution of (g) into about 0.5 ml unit doses; wherein the unit dose formulation has a molar ratio of starting albuterol free base: H₃PO₄ of about 0.80 to about 1.20, a pH of about 3.0 to about 4.5, and the unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.0 to about 4.5, if necessary. In an alternate embodiment, the process can optionally further comprise the addition of sodium phosphate dibasic in an amount sufficient to increase the pH of the unit dose formulation to about 3.0 to about 4.5, if necessary.

In some embodiments of the present invention, the unit dose formulation comprises a stabilizing agent selected from the group consisting of a chelating agent, a preservative, an antioxidant or a combination thereof.

In certain embodiments, the stabilizing agent is a chelating agent. In one embodiment, the chelating agent is EDTA.

In certain other embodiments of the present invention, the stabilizing agent is a preservative. In one embodiment, the preservative is benzalkonium chloride.

In yet other embodiments of the present invention, the stabilizing agent is an antioxidant. In certain embodiments, the unit dose formulation comprising a β₂ agonist further comprises an antioxidant selected from the group consisting of sodium ascorbate, sodium citrate, alpha tocopherol, or vitamin E.

In some embodiments of the present invention, the unit dose formulation further comprises a second pharmaceutically active agent. In one embodiment, the second pharmaceutically active agent is a corticosteroid. In another embodiment, the second pharmaceutically active agent is an antibiotic. In still another embodiment, the second pharmaceutically active agent is an anti-cholinergic agent. In yet another embodiment, the second pharmaceutically active agent is a dopamine (D2) receptor agonist.

In certain embodiments of the present invention, methods are provided for the treatment of a patient diagnosed with, or suspected of having, a bronchoconstrictive disorder, which comprise the administration of the unit dose formulation described herein. In some embodiments, the bronchoconstrictive disorder is selected from the group consisting of asthma, pediatric asthma, bronchial asthma, allergic asthma, occupational asthma, aspirin sensitive asthma, exercise-induced asthma, intrinsic asthma, chronic obstructive pulmonary disease (COPD), chronic bronchitis, cystic fibrosis and emphysema.

### INCORPORATION BY REFERENCE

Unless stated otherwise, all publications and patent applications mentioned in this specification are herein incorporated by reference to the same extent as if each individual publication or patent application was specifically and individually indicated to be incorporated by reference.

### BRIEF DESCRIPTION OF THE FIGURES

FIG. 1 provides a flowchart setting forth one embodiment of a large scale manufacturing process for the production and packaging of the approximately 0.5 ml unit dose formulations described herein.

FIG. 2 provides a flowchart setting forth a second embodiment of a large scale manufacturing process for the production and packaging of the approximately 0.5 ml unit dose formulations described herein.

### DETAILED DESCRIPTION OF THE INVENTION

Reference will now be made in detail to embodiments of the methods and unit dosage formulations disclosed herein. Examples of the embodiments are illustrated in the following Examples section.

Unless defined otherwise, all technical and scientific terms used herein have the same meaning as is commonly understood by one of skill in the art to which the inventions described herein belong. All patents and publications referred to herein are incorporated by reference.

Certain Definitions

As used herein, the terms “comprising,” “including,” “such as,” and “for example” are used in their open, non-limiting sense.

The term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 15%, which are also effective and safe.

“Albuterol” is an optically active compound which can exist as an (R)- or an (S)-enantiomer, or as a mixture of the two enantiomers. The term “albuterol” usually refers to a racemic mixture of both the (R)- and (S)-albuterol enantiomers. Herein, the term albuterol is defined as including a racemic mixture, a single enantiomer of albuterol, or any mixture of enantiomers of albuterol. Traditional racemic albuterol and racemic albuterol sulfate are commercially available as Proventil®, Ventolin® and Vormax®. The pure (R)-enantiomer, which has the generic name levalbuterol, is commercially available as Xopenex®. In addition, albuterol, as used herein, includes salbutamol, albuterol free base, as well as pharmaceutically acceptable salts of albuterol, including, but not limited to, hydrochloride, sulfate, maleate, tartrate, citrate, phosphate and the like. Certain exemplary salts are described in U.S. Pat. No. 3,644,353, which is incorporated herein by reference in its entirety.

“Levalbuterol” is a relatively selective beta2-adrenergic receptor agonist and is the (R)-enantiomer of the albuterol. Xopenex Inhalation Solution is supplied in unit-dose vials and requires no dilution before by nebulization. Each 3 mL unit-dose vial contains either 0.63 mg of levalbuterol (as 0.73 mg of levalbuterol HCl) or 1.25 mg of levalbuterol (as 1.44 mg of levalbuterol HCl), sodium chloride to adjust tonicity, and sulfuric acid to adjust the pH to 4.0 (3.3 to 4.5). Because levalbuterol consists essentially of the pure (R)-enantiomer of albuterol, it is hypothesized that the therapeutically effective dose of levalbuterol is approximately one-half the therapeutically effective dose of racemic albuterol.

“Bronchoconstrictive disorder,” as used herein, refers to any disorder or disease related to the reduction in the inner diameter of the bronchial pathway, e.g., a bronchus or bronchi, including, but not limited to, asthma, pediatric asthma, bronchial asthma, allergic asthma, occupational asthma, aspirin sensitive asthma, exercise-induced asthma, intrinsic asthma, chronic obstructive pulmonary disease (COPD), chronic bronchitis, cystic fibrosis and emphysema.

“Bronchodilation,” as used herein, refers to the expansion of the bronchial air passages to treat or prevent a bronchoconstrictive disorder.

“β₂ agonists” or “β₂ adrenergic receptor agonists,” as used herein, refers to any agent which can activate the β₂ adrenergic receptor. Short acting or long acting β₂ agonists are known in the art and include, but are not limited to, albuterol, terbutaline, bitolterol, levalbuterol, metaproterenol, pirbuterol, formoterol, arformoterol, salmeterol, or combinations thereof. In certain embodiments of the present invention, the unit dose formulations comprising β₂ agonists are sterile, thus eliminating the need for preservatives. In other embodiments, the unit dose formulations comprising β₂ agonists can comprise a stabilizing agent, e.g., a chelating agent, a preservative, or an antioxidant.

“Corticosteroids,” as used herein, refers to a group of drugs similar to the natural corticosteroid hormones produced by the cortex of the adrenal glands. Corticosteroids act to inhibit late phase allergic reactions via a variety of mechanisms, including decreasing the density of mast cells along mucosal surfaces, decreasing chemotaxis and activation of eosinophils, decreasing cytokine production by lymphocytes, monocytes, mast cells and eosinophils, inhibiting the metabolism of arachidonic acid and other mechanisms.

“Drug absorption” or “absorption” typically refers to the process of movement of drug from site of delivery of a drug across a barrier into a blood vessel or the site of action, e.g., a drug being absorbed in the pulmonary capillary beds of the alveoli.

“Inhalation nebulizer,” as used herein, refers to a device that turns medications into a fine mist for delivery to the lungs.

“Stabilizing agents,” as used herein, refers to any a chemical compound that is added to a unit dose formulation to protect against decay or decomposition. As used herein, stabilizing agents include chemical agents selected from the group of antimicrobials, antioxidants, chelating agents, complexing agents, and preservatives. In certain embodiments, stabilizing agents include, but are not limited to, edetate disodium (EDTA) or ethyleneglycol-bis(oxyethylenenitrilo)-tetraacetic acid (EGTA) and salts thereof, such as the disodium salt, citric acid, nitrilotriacetic acid, benzalkonium chloride (BAC) or benzoic acid, benzoates such as sodium benzoate, vitamins and vitamin esters, provitamins, ascorbic acid, vitamin E, and combinations thereof.

A “therapeutically effective amount” or “effective amount” is that amount of a pharmaceutical agent to achieve a pharmacological effect. The term “therapeutically effective amount” includes, for example, a prophylactically effective amount. An “effective amount” of a β₂ agonist, such as albuterol, is an amount effective to achieve a desired pharmacologic effect or therapeutic improvement without undue adverse side effects. The effective amount of a β₂ agonist, such as albuterol, will be selected by those skilled in the art depending on the particular patient and the disease level. It is understood that “an effect amount” or “a therapeutically effective amount” can vary from subject to subject, due to variation in metabolism of a β₂ agonist, such as albuterol, age, weight, general condition of the subject, the condition being treated, the severity of the condition being treated, and the judgment of the prescribing physician.

“Treat” or “treatment” as used in the context of a bronchoconstrictive disorder refers to any treatment of a disorder or disease related to the constriction of the bronchi, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent.”

I. Unit Dose Formulations of the Present Invention

In certain embodiments of the present invention, unit dose formulations are provided which comprise a therapeutically effective amount of a β₂ agonist solution, wherein the unit dose formulation is prepared by the process comprising the steps of: (a) providing from about 0.80 to about 0.9 weight % of a tonicity adjusting agent; (b) mixing the tonicity adjusting agent with approximately 80 weight % water to form an aqueous solution; (c) adding from about 0.015 to about 0.30 weight β₂ agonist as a free base to the solution of (b); (d) adding from about 0.01 to about 1.3 weight % of a pH adjusting agent to the solution of (c); (e) adding water to the solution of (d) in a quantity sufficient to provide a total weight % equal to 100; (f) filter sterilizing the solution of (e); and (g) dividing the solution of (f) into about 0.5 ml unit doses. In certain embodiments, the unit dose formulation comprises a molar ratio of starting 32 agonist free base: pH adjusting agent of about 0.8 to about 1.20 and a pH of about 3.0 to about 4.5. In other embodiments, the unit dose formulation comprises a molar ratio of starting β₂ agonist free base: pH adjusting agent of about 0.85 to about 1.10 and a pH of about 3.5 to about 4.0. In still other embodiments, the process can optionally further comprise the addition of a second pH adjusting agent, e.g., NaOH, in an amount sufficient to increase the pH of the unit dose formulation to a desired pH, for example, to obtain a pH from about 3.0 to about 4.5, if necessary. In one embodiment, the unit dose formulation is prepared using from about 0.03 to about 0.30 weight % albuterol. In another embodiment, the unit dose formulation is prepared using from about 0.015 to about 0.15% weight % levalbuterol.

In other embodiments of the present invention, unit dose formulations are provided which comprise a therapeutically effective amount of a β₂ agonists solution, wherein the unit dose formulation is prepared by the process comprising the steps of: (a) providing from about 0.80 to about 0.9 weight % of a tonicity adjusting agent; (b) mixing the tonicity adjusting agent with approximately 80 weight % water to form an aqueous solution; (c) adding from about 0.015 to about 0.30 weight β₂ agonists as a free base to the solution of (b); (d) adding about 0.001 to about 5.0 weight % of a stabilizing agent to the solution of (c); (e) adding from about 0.01 to about 1.3 weight % of a pH adjusting agent to the solution of (d); (f) adding water to the solution of (e) in a quantity sufficient to provide a total weight % equal to 100; (g) filter sterilizing the solution of (f); and (h) dividing the solution of (g) into about 0.5 ml unit doses. In certain embodiments, the unit dose formulation comprises a molar ratio of starting β₂ agonist free base: pH adjusting agent of about 0.8 to about 1.20 and a pH of about 3.0 to about 4.5. In other embodiments, the unit dose formulation comprises a molar ratio of starting β₂ agonist free base: pH adjusting agent of about 0.85 to about 1.10 and a pH of about 3.5 to about 4.0. In still other embodiments, the process can optionally further comprise the addition of a second pH adjusting agent, e.g., NaOH, in an amount sufficient to increase the pH of the unit dose formulation to a desired pH, for example, to obtain a pH from about 3.0 to about 4.5, if necessary. In one embodiment, the unit dose formulation is prepared using from about 0.03 to about 0.30 weight % albuterol. In another embodiment, the unit dose formulation is prepared using from about 0.015 to about 0.15 weight % levalbuterol.

β₂ agonists useful in the unit dose formulations of the present invention include, but are not limited to, short acting β₂ agonists selected from the group consisting of albuterol, terbutaline, bitolterol, levalbuterol, metaproterenol, pirbuterol, and combinations thereof. In certain embodiments, the short acting β₂ agonist is selected from the group consisting of albuterol, terbutaline, levalbuterol, pirbuterol, and combinations thereof. In one embodiment, the short acting β₂ agonist is albuterol. In another embodiment, the short acting β₂ agonist is levalbuterol.

Tonicity adjusting agents useful in unit dose formulations of the present invention include, but are not limited to, sodium chloride, potassium chloride, mannitol, dextrose, glycerin, and propylene glycol. In one embodiment, the tonicity adjusting agent is sodium chloride.

pH adjusting agents useful in unit dose formulations of the present invention include, but are not limited to, hydrochloric acid (HCl), citric acid or phosphoric acid (H₃PO₄). In certain embodiments, a second pH adjusting agent can optionally be added to increase the pH of the unit dose formulations, if necessary. By way of a non-limiting example, such second pH adjusting agents can include NaOH, sodium citrate, sodium phosphate monobasic or sodium phosphate dibasic, and are useful in the process of the present invention in amounts sufficient to provide the unit dose formulation a desired pH, for example, to obtain a pH from about 3.0 to about 4.5. In still other embodiments, suitable pH buffering systems can be used in the unit dose formulations to maintain the pH of the unit dose formulation within a desired range and include, but are not limited to, sodium citrate/citric acid, sodium acetate/acetic acid, sodium or potassium phosphate dibasic/monobasic, and any other pharmaceutically acceptable pH buffering agent(s) known in the art.

Stabilizing agents useful in the unit dose formulations of the present invention include any a chemical compound that is added to a unit dose formulation to protect against decay or decomposition and includes chelating agents, preservatives, antioxidants, and complexing agents.

In certain embodiments, the stabilizing agent is a chelating agent. Chelating agents suitable for use in the unit dose formulations of the present invention include, but are not limited to, edetate disodium (EDTA) or ethyleneglycolbis(oxyethylenenitrilo)-tetraacetic acid (EGTA) and salts thereof, such as the disodium salt, citric acid, nitrilotriacetic acid. In one embodiment, the unit dose formulation comprising a β₂ agonist further comprises edetate disodium (EDTA).

In certain other embodiments, the stabilizing agent is a preservative. Suitable preservatives for use in the unit dose formulations of the present invention include, but are not limited to, benzalkonium chloride (BAC), benzoic acid, or benzoates such as sodium benzoate. In one embodiment, the unit dose formulation comprising a β₂ agonist further comprises benzalkonium chloride (BAC).

In other embodiments, the stabilizing agent is an antioxidant. Suitable antioxidants for use in the unit dose formulations of the present invention include, but are not limited to, one or more of the following: butylated hydroxyanisole (BHA), butylated hydroxytoluene (BHT), ascorbic acid, ascorbyl palmitate, propyl gallate, dodecyl gallate, ethyl gallate, octyl gallate, alpha tocopherol, sodium ascorbate, sodium citrate, sodium metabisulfite, fumaric acid, malic acid, vitamins and vitamin esters, provitamins, ascorbic acid, vitamin E, and any pharmaceutically compatible antioxidant known in the art, and combinations thereof. In certain embodiments, the unit dose formulation comprising a β₂ agonist further comprises an antioxidant selected from the group consisting of sodium ascorbate, sodium citrate, alpha tocopherol, or vitamin E.

In other embodiments of the present invention, unit dose formulations are provided which comprise a therapeutically effective amount of an albuterol solution, wherein the unit dose formulation is prepared by the process comprising the steps of: (a) providing from about 0.84 to about 0.90 weight % of NaCl; (b) mixing the NaCl with approximately 80 weight % water to form an aqueous solution; (c) adding from about 0.03 to about 0.25 weight % albuterol free base to the solution of (b); (d) adding from about 0.10 to about 1.3 weight % of HCl to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: HCl of about 0.85 to about 1.20, a pH of about 3.0 to about 4.5, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.0 to about 4.5, if necessary.

In one embodiment, the unit dose formulation comprises therapeutically effective amount of an albuterol solution, wherein the unit dose formulation is prepared by the process comprising the steps of: (a) providing 0.90 weight % of NaCl; (b) mixing the NaCl with approximately 80 weight % water to form an aqueous solution; (c) adding about 0.03 weight % albuterol free base to the solution of (b); (d) adding about 0.17 weight % of HCl to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: HCl of about 0.87, a pH of about 3.7, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary.

In another embodiment, the unit dose formulation comprises therapeutically effective amount of an albuterol solution, wherein the unit dose formulation is prepared by the process comprising the steps of: (a) providing about 0.87 weight % of NaCl; (b) mixing the NaCl with approximately 80 weight % water to form an aqueous solution; (c) adding about 0.12 weight % albuterol free base to the solution of (b); (d) adding about 0.55 weight % of HCl to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: HCl of about 1.08, a pH of about 3.6, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.6, if necessary.

In yet another embodiment, the unit dose formulation comprises therapeutically effective amount of an albuterol solution, wherein the unit dose formulation is prepared by the process comprising the steps of: (a) providing about 0.84 weight % of NaCl; (b) mixing the NaCl with approximately 80 weight % water to form an aqueous solution; (c) adding about 0.25 weight % albuterol free base to the solution of (b); (d) adding about 1.06 weight % of HCl to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: HCl of about 1.16, a pH of about 3.4, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.4, if necessary.

In certain other embodiments of the present invention, unit dose formulations are provided which comprise a therapeutically effective amount of an albuterol solution, wherein the unit dose formulation is prepared by the process comprising the steps of: (a) providing from about 0.84 to about 0.90 weight % of NaCl; (b) mixing the NaCl with approximately 80 weight % water to form an aqueous solution; (c) adding from about 0.03 to about 0.25 weight % albuterol free base to the solution of (b); (d) adding from about 0.03 to about 0.35 weight % of citric acid to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: citric acid of about 0.80 to about 1.0, a pH of about 3.0 to about 4.5, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.0 to about 4.5, if necessary. In an alternate embodiment, the process can optionally further comprise the addition of sodium citrate in an amount sufficient to increase the pH of the unit dose formulation to about 3.0 to about 4.5, if necessary.

In one embodiment, the unit dose formulation comprises therapeutically effective amount of an albuterol solution, wherein the unit dose formulation is prepared by the process comprising the steps of: (a) providing 0.90 weight % of NaCl; (b) mixing the NaCl with approximately 80 weight % water to form an aqueous solution; (c) adding about 0.03 weight % albuterol free base to the solution of (b); (d) adding about 0.03 weight % of citric acid to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: citric acid of about 0.80, a pH of about 3.7, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium citrate in an amount sufficient to increase the pH of the unit dose formulation to about 3.7, if necessary.

In another embodiment, the unit dose formulation comprises therapeutically effective amount of an albuterol solution, wherein the unit dose formulation is prepared by the process comprising the steps of: (a) providing about 0.87 weight % of NaCl; (b) mixing the NaCl with approximately 80 weight % water to form an aqueous solution; (c) adding about 0.12 weight % albuterol free base to the solution of (b); (d) adding about 0.11 weight % of citric acid to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: citric acid of about 0.88, a pH of about 3.7, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium citrate in an amount sufficient to increase the pH of the unit dose formulation to about 3.7, if necessary.

In yet another embodiment, the unit dose formulation comprises therapeutically effective amount of an albuterol solution, wherein the unit dose formulation is prepared by the process comprising the steps of: (a) providing about 0.84 weight % of NaCl; (b) mixing the NaCl with approximately 80 weight % water to form an aqueous solution; (c) adding about 0.25 weight % albuterol free base to the solution of (b); (d) adding about 0.21 weight % of citric acid to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: citric acid of about 0.96, a pH of about 3.7, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium citrate in an amount sufficient to increase the pH of the unit dose formulation to about 3.7, if necessary.

In other embodiments of the present invention, unit dose formulations are provided which comprise a therapeutically effective amount of an albuterol solution, wherein the unit dose formulation is prepared by the process comprising the steps of: (a) providing from about 0.84 to about 0.90 weight % of NaCl; (b) mixing the NaCl with approximately 80 weight % water to form an aqueous solution; (c) adding from about 0.03 to about 0.25 weight % albuterol free base to the solution of (b); (d) adding from about 0.10 to about 1.20 weight % of H₃PO₄ to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: H₃PO₄ of about 0.80 to about 1.20, a pH of about 3.0 to about 4.5, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.0 to about 4.5, if necessary. In an alternate embodiment, the process can optionally further comprise the addition of sodium phosphate dibasic in an amount sufficient to increase the pH of the unit dose formulation to about 3.0 to about 4.5, if necessary.

In one embodiment, the unit dose formulation comprises therapeutically effective amount of an albuterol solution, wherein the unit dose formulation is prepared by the process comprising the steps of: (a) providing 0.90 weight % of NaCl; (b) mixing the NaCl with approximately 80 weight % water to form an aqueous solution; (c) adding about 0.03 weight % albuterol free base to the solution of (b); (d) adding about 0.10 weight % of H₃PO₄ to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: H₃PO₄ of about 0.80 to about 1.20, a pH of about 3.7, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium phosphate dibasic in an amount sufficient to increase the pH of the unit dose formulation to about 3.7, if necessary.

In another embodiment, the unit dose formulation comprises therapeutically effective amount of an albuterol solution, wherein the unit dose formulation is prepared by the process comprising the steps of: (a) providing about 0.87 weight % of NaCl; (b) mixing the NaCl with approximately 80 weight % water to form an aqueous solution; (c) adding about 0.12 weight % albuterol free base to the solution of (b); (d) adding about 0.34 weight % of H₃PO₄ to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: H₃PO₄ of about 0.80 to about 1.20, a pH of about 3.7, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium phosphate dibasic in an amount sufficient to increase the pH of the unit dose formulation to about 3.7, if necessary.

In yet another embodiment, the unit dose formulation comprises therapeutically effective amount of an albuterol solution, wherein the unit dose formulation is prepared by the process comprising the steps of: (a) providing about 0.84 weight % of NaCl; (b) mixing the NaCl with approximately 80 weight % water to form an aqueous solution; (c) adding about 0.25 weight % albuterol free base to the solution of (b); (d) adding about 1.02 weight % of H₃PO₄ to the solution of (c); (e) adding water to the solution of (d) in a quantity sufficient to provide a total weight % equal to 100; (f) filter sterilizing the solution of (e); and (g) dividing the solution of (f) into about 0.5 ml unit doses, wherein said unit dose formulation has a molar ratio of starting albuterol free base: H₃PO₄ of about 0.80 to about 1.20, a pH of about 3.7, and wherein said unit dose formulation comprises a therapeutically effective amount of albuterol and is suitable for inhalation therapy via nebulization. In addition, the process can optionally further comprise the addition of NaOH in an amount sufficient to increase the pH of the unit dose formulation to about 3.7, if necessary. In an alternate embodiment, the process can optionally further comprise the addition of sodium phosphate dibasic in an amount sufficient to increase the pH of the unit dose formulation to about 3.7, if necessary.

In certain embodiments, the unit dose formulations described herein can be used in the treatment of a patient diagnosed with, or suspected of having, a disease selected from the group consisting of asthma, pediatric asthma, bronchial asthma, allergic asthma, occupational asthma, aspirin sensitive asthma, exercise-induced asthma, intrinsic asthma, chronic obstructive pulmonary disease (COPD), chronic bronchitis, cystic fibrosis and emphysema.

II. Combination Therapies Comprising the Unit Dose Formulations of the Present Invention

In certain embodiments, the unit dose formulations of the present invention can further comprise a second pharmaceutically active agent. In certain embodiments, the second pharmaceutically active agent can be selected from (a) a corticosteroid; (b) an antibiotic; (c) an anti-cholinergic agent; or (d) a dopamine (D₂) receptor agonist.

Corticosteriods for use in combination therapy with the unit dose formulations described herein include, but are not limited to, aldosterone, beclomethasone, betamethasone, budesonide, ciclesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, mometasone, paramethasone, prednisolone, prednisone, rofleponide, RPR 106541, tixocortol, triamcinolone, and their respective pharmaceutically acceptable derivatives. In one embodiment, the second pharmaceutically active agent is budesonide.

Antibiotics for use in combination therapy with the unit dose formulations described herein include, but are not limited to, penicillins, cephalosporins, macrolides, sulfonamides, aminoglycosides, and β-lactam antibiotics.

Anticholinergic agents for use in combination therapy with the unit dose formulations described herein include, but are not limited to, ipratropium bromide, oxitropium bromide, atropine methyl nitrate, atropine sulfate, ipratropiurn, belladonna extract, scopolamine, scopolamine methobromide, homatropine methobromide, hyoscyamine, isopriopramide, orphenadrine, tiotropium bromide and glycopyrronium bromide.

Dopamine (D₂) receptor agonists for use in combination therapy with the unit dose formulations described herein include, but are not limited to, Apomorphine ((r)-5,6,6a,7-tetrahydro-6-methyl-4H-dibenzo-quinoline-10,11-diol); Bromocriptine ((5′α)-2-bromo-12′-hydroxy-2′-(1-methylethyl)-5′-(2-methylpropyl)erg otaman-3′,6′,18-trione); Cabergoline ((8β)-N-(3(dimethylamino)propyl)-N-((ethylamino)carbonyl)-6-(2-propenyl)ergoline-8-carboxamide); Lisuride (N′-((8α)-9,10-didehydro-6-methylergolin-8-yl)-N,N-diethylurea); Pergolide ((8β)-8-((methylthio)methyl)-6-propylergoline); Levodopa (3-hydroxy-L-tryrosine); Pramipexole ((s)-4,5,6,7-tetrahydro-N₆-propyl-2,6-benzothiazolediamine); Quinpirole hydrochirodie (trans-(−)-4-aR-4,4a,5,6,7,8,8a,9-octahydro-5-propyl-1H-pyrazolo[3,4-g]quinoline hydrochloride); Ropinirole (4-(2-(dipropylamino)ethyl)-1,3-dihydro-2H-indol-2-one); and Talipexole (5,6,7,8-tetrahydro-6-(2-propenyl)-4H-thiazolo[4,5-d]azepin-2-amine). Other dopamine D₂ receptor agonists for use herein are disclosed in International Patent Application Publication No. WO 99/36095.

Other active ingredients for use in the inhalable compositions described herein include, but are not limited to, IL-5 inhibitors such as those disclosed in U.S. Pat. No. 5,668,110, No. 5,683,983, No. 5,677,280, No. 6,071,910 and No. 5,654,276, each of which is incorporated by reference herein; anti-sense modulators of IL-5 such as those disclosed in U.S. Pat. No. 6,136,603, the relevant disclosure of which is hereby incorporated by reference; milrinone (1,6-dihydro-2-methyl-6-oxo-[3,4′-bipyridine]-5-carbonitrile); milrinone lactate; tryptase inhibitors such as those disclosed in U.S. Pat. No. 5,525,623, which is incorporated by reference herein; tachykinin receptor antagonists such as those disclosed in U.S. Pat. No. 5,691,336, No. 5,877,191, No. 5,929,094, No. 5,750,549 and No. 5,780,467, each of which is incorporated by reference herein; leukotriene receptor antagonists such as montelukast sodium (Singular, R-(E)]-1-[[[1-[3-[2-(7-chloro-2-quinolinyl)-ethenyl]-phenyl]-3-[2-(1-hydroxy-1-methylethyl)-phenyl]-propyl]-thio]-methyl]cyclopro-paneacetic acid, monosodium salt), 5-lypoxygenase inhibitors such as zileuton (Zyflo®, Abbott Laboratories, Abbott Park, Ill.), and anti-IgE antibodies such as Xolair (recombinant humanized anti-IgE monoclonal antibody (CGP 51901; IGE 025A; rhuMAb-E25), Genentech, Inc., South San Francisco, Calif.), and topical anesthetics such as lidocaine, N-arylamide, aminoalkylbenzoate, prilocalne, etidocaine (U.S. Pat. No. 5,510,339, No. 5,631,267, and No. 5,837,713, the relevant disclosures of which are hereby incorporated by reference).

Methods of treatment using combination therapy is provided herein, wherein the unit dose formulations described herein and a second pharmaceutically active agent can be administered together in the treatment of a patient diagnosed with, or suspected of having, a disease selected from the group consisting of asthma, pediatric asthma, bronchial asthma, allergic asthma, occupational asthma, aspirin sensitive asthma, exercise-induced asthma, intrinsic asthma, chronic obstructive pulmonary disease (COPD), chronic bronchitis, cystic fibrosis and emphysema.

In some embodiments, the combination therapy comprises a unit dose formulation of the present invention and a second pharmaceutically active agent wherein the β₂ agonist and a second pharmaceutically active agent are formulated as one unit dose solution to be delivered simultaneously. In other embodiments, the combination therapy can comprise a unit dose formulation of the present invention and an inhalation mixture comprising a second pharmaceutically active agent wherein the two inhalation mixtures are independently prepared, mixed at the time of delivery, and simultaneously delivered. In other embodiments, the combination therapy comprises a unit dose formulation of the present invention and an inhalation mixture comprising a second pharmaceutically active agent wherein the two inhalation mixtures are consecutively delivered.

III. Nebulizers for Use in the Delivery of the Unit Dose Formulations of the Present Invention

The unit dose formulations described herein are suitable for the treatment of a disease or disorder through inducement of bronchodilation in a patient in need thereof upon delivery of one or more of the unit dose formulations via an inhalation nebulizer. Any known inhalation nebulizer is suitable for use in the present invention. Suitable inhalation nebulizers include, e.g., jet nebulizers, ultrasonic nebulizers, pulsating nebulizers, and nebulizers comprising a vibrating mesh or plate with an aqueous chamber (e.g., Pari eFlow®, TouchSpray®, AeroNeb® Aerodose Inhaler, or Omron® NE-U03 NE-U22). Any of these and other known nebulizers can be used to deliver the inhalation mixtures described in the present invention. In some embodiments, the nebulizers are available from, e.g., Pari GmbH (Stamberg, Germany), DeVilbiss Healthcare (Heston, Middlesex, UK), Healthdyne, Vital Signs, Baxter, Allied Health Care, Invacare, Hudson, Omron, Bremed, AirSep, Luminscope, Medisana, Siemens, Aerogen, Mountain Medical, Aerosol Medical Ltd. (Colchester, Essex, UK), AFP Medical (Rugby, Warwickshire, UK), Bard Ltd. (Sunderland, UK), Carri-Med Ltd. (Dorking, UK), Plaem Nuiva (Brescia, Italy), Henleys Medical Supplies (London, UK), Intersurgical (Berkshire, UK), Lifecare Hospital Supplies (Leies, UK), Medic-Aid Ltd. (West Sussex, UK), Medix Ltd. (Essex, UK), Sinclair Medical Ltd. (Surrey, UK), and many others. In certain embodiments, the nebulizer comprises a vibrating mesh or plate with an aqueous chamber. In one embodiment, the nebulizer is a Pari eFlow® nebulizer.

Other nebulizers suitable for use in the methods and systems describe herein include, but are not limited to, jet nebulizers (optionally sold with compressors), ultrasonic nebulizers, and others. Exemplary jet nebulizers for use herein include Pari LC plus/ProNeb, Pari LC plus/ProNeb Turbo, Pari LCPlus/Dura Neb 1000 & 2000 Pari LC plus/Walkhaler, Pari LC plus/Pari Master, Pari LC star, Omron CompAir XL Portable Nebulizer System (NE-C18 and JetAir Disposable nebulizer), Omron compare Elite Compressor Nebulizer System (NE-C21 and Elite Air Reusable Nebulizer, Pari LC Plus or Pari LC Star nebulizer with Proneb Ultra compressor, Pulomo-aide, Pulmo-aide LT, Pulmo-aide traveler, Invacare Passport, Inspiration Healthdyne 626, Pulmo-Neb Traveler, DeVilbiss 646, Whisper Jet, AcornII, Misty-Neb, Allied aerosol, Schuco Home Care, Lexan Plasic Pocet Neb, SideStream Hand Held Neb, Mobil Mist, Up-Draft, Up-DraftII, T Up-Draft, ISO-NEB, Ava-Neb, Micro Mist, and PulmoMate.

Exemplary ultrasonic nebulizers for use herein include MicroAir, UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Lumiscope's Ultrasonic Nebulizer, Medisana Ultrasonic Nebulizer, Microstat Ultrasonic Nebulizer, and Mabismist Hand Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, Aeroneb Portable Nebulizer System, Aerodose Inhaler, and AeroEclipse Breath Actuated Nebulizer.

In certain embodiments, nebulizers suitable for use in the presently described invention include nebulizers comprising a vibrating mesh or plate with an aqueous chamber. Such nebulizers are sold commercially as, e.g., Pari eFlow®, and are described in U.S. Pat. Nos. 6,962,151, 5,518,179, 5,261,601, and 5,152,456, each of which is specifically incorporated by reference herein. In still other embodiments, suitable nebulizers for use in the presently described include nebulizers comprising a vibrating mesh or plate with multiple apertures as described by R. Dhand in New Nebuliser Technology—Aerosol Generation by Using a Vibrating Mesh or Plate with Multiple Apertures, Long-Term Healthcare Strategies 2003, (July 2003), and p. 1-4 and Respiratory Care, 47: 1406-1416 (2002), the entire disclosure of each of which is hereby incorporated by reference.

The parameters used in nebulization, such as flow rate, mesh membrane size, aerosol inhalation chamber size, mask size and materials, valves, and power source may be varied in accordance with the principles of the present invention to maximize their use with different types of inhalation mixtures or different types of β₂ agonists and delivery time conditions specified herein.

### EXAMPLES

The following ingredients, processes and procedures for practicing the methods described herein correspond to that described above. Any methods or materials not particularly described in the following examples are within the scope of the invention and will be apparent to those skilled in the art with reference to the disclosure herein.

### Example 1

Albuterol inhalation solutions were prepared from albuterol free base with varying concentrations according to the following specifications:

| | Albuterol Concentration [mg/dose] | | |
|---|---|---|---|
| | 0.15 | 0.60 | 1.25 |
| Ingredient | LOW | MEDIUM | HIGH |
| Albuterol free base (weight %) | 0.03 | 0.12 | 0.25 |
| Hydrochloric acid (1 N) | ad ˜pH 3.75 | ad ˜pH 3.75 | ad ˜pH 3.75 |
| NaCl | ad ˜0.290 Osmol/kg | ad ˜0.290 Osmol/kg | ad ˜0.290 Osmol/kg |
| Water (weight %) | qs to 100 | qs to 100 | qs to 100 |

Three albuterol inhalation solutions (LOW, MEDIUM, and HIGH) having a total mass of approximately 100 grams were prepared using the following pharmaceutically acceptable agents:

| | Albuterol Concentration [mg/dose] | | |
|---|---|---|---|
| | 0.15 | 0.60 | 1.25 |
| Ingredient | LOW | MEDIUM | HIGH |
| Albuterol free base (weight %) | 0.03 | 0.12 | 0.25 |
| Hydrochloric acid (1 N) (weight %) | 0.17 | 0.55 | 1.06 |
| NaCl (weight %) | 0.90 | 0.87 | 0.84 |
| Water (weight %) | qs to 100 | qs to 100 | qs to 100 |

The LOW concentration albuterol inhalation solution was formulated according to the following methods: (a) 902 mg NaCl was added to approximately 80 g water to form an aqueous solution; (b) 30 mg albuterol free base was added to the solution of (a); (c) 170 mg HCl-1N was added to the solution of (b); and (d) approximately 20 g water was added to the solution of (c). The albuterol inhalation solution prepared according to this method has a pH=3.73 and an osmolality=0.288 Osmol/kg. The molar ratio of starting albuterol free base: hydrochloric acid was 0.871.

The MEDIUM concentration albuterol inhalation solution was formulated according to the following methods: (a) 875 mg NaCl was added to approximately 80 g water to form an aqueous solution; (b) 60 mg albuterol free base was added to the solution of (a); (c) 550 mg HCl-1N was added to the solution of (b); and (d) approximately 20 g water was added to the solution of (c). The albuterol inhalation solution prepared according to this method has a pH=3.60 and an osmolality=0.286 Osmol/kg. The molar ratio of starting albuterol free base: hydrochloric acid was 1.076.

The HIGH concentration albuterol inhalation solution was formulated according to the following methods: (a) 840 mg NaCl was added to approximately 80 g water to form an aqueous solution; (b) 250 mg albuterol free base was added to the solution of (a); (c) 1060 mg HCl-1N was added to the solution of (b); and (d) approximately 20 g water was added to the solution of (c). The albuterol inhalation solution prepared according to this method has a pH=3.41 and an osmolality=0.282 Osmol/kg. The molar ratio of starting albuterol free base: hydrochloric acid was 1.164.

The three concentrations of the albuterol inhalation solutions are filter sterilized and packaged into 0.5 ml unit doses by adding about 0.5 to about 0.58 mls of the albuterol solutions into polyethylene unit dose vials. The resulting unit dose formulations will have a concentration of approximately 0.15 mg starting albuterol free base/dose, approximately 0.60 mg starting albuterol free base/dose, and approximately 1.25 mg starting albuterol free base/dose, respectively.

### Example 2

Albuterol inhalation solutions were prepared from albuterol free base with varying concentrations according to the following specifications:

| | Albuterol Concentration [mg/dose] | | |
|---|---|---|---|
| | 0.15 | 0.60 | 1.25 |
| Ingredient | LOW | MEDIUM | HIGH |
| Albuterol free base (weight %) | 0.03 | 0.12 | 0.25 |
| Citric acid (anhydrous) | ad ˜pH 3.75 | ad ˜pH 3.75 | ad ˜pH 3.75 |
| NaCl | ad ˜0.290 Osmol/kg | ad ˜0.290 Osmol/kg | ad ˜0.290 Osmol/kg |
| Water (weight %) | qs to 100 | qs to 100 | qs to 100 |

Three albuterol inhalation solutions (LOW, MEDIUM, and HIGH) having a total mass of approximately 100 grams were prepared using the following pharmaceutically acceptable agents:

| | Albuterol Concentration [mg/dose] | | |
|---|---|---|---|
| | 0.15 | 0.60 | 1.25 |
| Ingredient | LOW | MEDIUM | HIGH |
| Albuterol free base (weight %) | 0.03 | 0.12 | 0.25 |
| Citric acid (anhydrous) (weight %) | 0.03 | 0.11 | 0.21 |
| NaCl (weight %) | 0.90 | 0.87 | 0.84 |
| Water (weight %) | qs to 100 | qs to 100 | qs to 100 |

The LOW concentration albuterol inhalation solution was formulated according to the following methods: (a) 900 mg NaCl was added to approximately 80 g water to form an aqueous solution; (b) 30 mg albuterol free base was added to the solution of (a); (c) 30 mg citric acid was added to the solution of (b); and (d) approximately 20 g water was added to the solution of (c). The albuterol inhalation solution prepared according to this method has a pH=3.76 and an osmolality=0.286 Osmol/kg. The molar ratio of starting albuterol free base: citric acid was 0.803.

The MEDIUM concentration albuterol inhalation solution was formulated according to the following methods: (a) 871 mg NaCl was added to approximately 80 g water to form an aqueous solution; (b) 60 mg albuterol free base was added to the solution of (a); (c) 110 mg citric acid was added to the solution of (b); and (d) approximately 20 g water was added to the solution of (c). The albuterol inhalation solution prepared according to this method has a pH=3.76 and an osmolality=0.286 Osmol/kg. The molar ratio of starting albuterol free base: citric acid was 0.876.

The HIGH concentration albuterol inhalation solution was formulated according to the following methods: (a) 840 mg NaCl was added to approximately 80 g water to form an aqueous solution; (b) 250 mg albuterol free base was added to the solution of (a); (c) 210 mg citric acid was added to the solution of (b); and (d) approximately 20 g water was added to the solution of (c). The albuterol inhalation solution prepared according to this method has a pH=3.78 and an osmolality=0.286 Osmol/kg. The molar ratio of starting albuterol free base: citric acid was 0.956.

The three concentrations of the albuterol inhalation solutions are filter sterilized and packaged into 0.5 ml unit doses by adding about 0.5 to about 0.58 mls of the albuterol solutions into polyethylene unit dose vials. The resulting unit dose formulations will have a concentration of approximately 0.15 mg starting albuterol free base/dose, approximately 0.60 mg starting albuterol free base/dose, and approximately 1.25 mg starting albuterol free base/dose, respectively.

### Example 3

Albuterol inhalation solutions were prepared from albuterol free base with varying concentrations according to the following specifications:

| | Albuterol Concentration [mg/dose] | | |
|---|---|---|---|
| | 0.15 | 0.60 | 1.25 |
| Ingredient | LOW | MEDIUM | HIGH |
| Albuterol free base (weight %) | 0.03 | 0.12 | 0.25 |
| H₃PO₄ (dilute) | ad ˜pH 3.75 | ad ˜pH 3.75 | ad ˜pH 3.75 |
| NaCl | ad ˜0.290 Osmol/kg | ad ˜0.290 Osmol/kg | ad ˜0.290 Osmol/kg |
| Water (weight %) | qs to 100 | qs to 100 | qs to 100 |

Three albuterol inhalation solutions (LOW, MEDIUM, and HIGH) having a total mass of approximately 100 grams were prepared using the following pharmaceutically acceptable agents:

| | Albuterol Concentration [mg/dose] | | |
|---|---|---|---|
| | 0.15 | 0.60 | 1.25 |
| Ingredient | LOW | MEDIUM | HIGH |
| Albuterol free base (weight %) | 0.03 | 0.12 | 0.25 |
| H₃PO₄ (dilute) (weight %) | 0.17 | 0.55 | 1.06 |
| NaCl (weight %) | 0.90 | 0.87 | 0.84 |
| Water (weight %) | qs to 100 | qs to 100 | qs to 100 |

The LOW concentration albuterol inhalation solution was formulated according to the following methods: (a) 901 mg NaCl was added to approximately 80 g water to form an aqueous solution; (b) 30 mg albuterol free base was added to the solution of (a); (c) 100 mg H₃PO₄ was added to the solution of (b); and (d) approximately 20 g water was added to the solution of (c). The albuterol inhalation solution prepared according to this method had a pH=3.72, and an osmolality=0.288 Osmol/kg.

The MEDIUM concentration albuterol inhalation solution was formulated according to the following methods: (a) 873 mg NaCl was added to approximately 80 g water to form an aqueous solution; (b) 60 mg albuterol free base was added to the solution of (a); (c) 340 mg H₃PO₄ was added to the solution of (b); and (d) approximately 20 g water was added to the solution of (c). The albuterol inhalation solution prepared according to this method had a pH=3.77 and an osmolality=0.285 Osmol/kg.

The HIGH concentration albuterol inhalation solution was formulated according to the following methods: (a) 840 mg NaCl was added to approximately 80 g water to form an aqueous solution; (b) 250 mg albuterol free base was added to the solution of (a); (c) 1020 mg H₃PO₄ was added to the solution of (b); and (d) approximately 20 g water was added to the solution of (c). The albuterol inhalation solution prepared according to this method had a pH=3.67 and an osmolality=0.283 Osmol/kg.

The three concentrations of the albuterol inhalation solutions are filter sterilized and packaged into 0.5 ml unit doses by adding about 0.5 to about 0.58 mls of the albuterol solutions into polyethylene unit dose vials. The resulting unit dose formulations will have a concentration of approximately 0.15 mg starting albuterol free base/dose, approximately 0.60 mg starting albuterol free base/dose, and approximately 1.25 mg starting albuterol free base/dose, respectively.

### Example 4

A patient experiencing asthma initiates treatment for asthma by inducing bronchodilation. The patient induces bronchodilation by placing a single unit dose of an inhalation mixture comprising about 1.25 mg/dose of albuterol as described in Example 1 into the reservoir of a Pari eFlow® vibrating membrane inhalation nebulizer. The delivery of the inhalation mixture by the nebulizer is then initiated. Over the course of less than about three (3) minutes, the inhalation mixture is delivered with the inhalation nebulizer and the symptoms of asthma are ameliorated or relieved.

### Example 5

The same procedure is followed as in Example 4; however, in this case the symptoms of the patient are not sufficiently ameliorated after the initial dose is delivered over the course of less than about three (3) minutes. In response to the continued presence of the symptoms, the patient repeats the procedure as set forth in Example 4. Upon the completion of the second delivery of the inhalation mixture of albuterol, the symptoms of asthma are ameliorated or relieved.

### Example 6

Albuterol inhalation solutions were prepared according to the methods set forth in Examples 1-3. The resulting albuterol solutions were combined with an equal volume of CBIS (Captisol Budesonide Inhalation Solution) to determine the chemical compatibility of the two solutions. The pH of the solutions is determined at time points 0 h, 0.5 h, 1 h, and 3 h. The appearance of the solution is noted during the observation period.

| Albuterol | | | | | | |
|---|---|---|---|---|---|---|
| Starting | | pH | | | | |
| Material | Acid | 0 h* | 0.5 h | 1 h | 3 h | Appearance |
| 0.03 | 1N—HCl | 4.40 | 4.33 | 4.40 | 4.32 | Clear, colorless, free of visible particle |
| | | | | | | during the entire observation time. |
| | H₃PO₄ | 4.36 | 4.32 | 4.38 | 4.33 | Clear, colorless, free of visible particle |
| | dilute | | | | | during the entire observation time. |
| | Citric acid | 4.16 | 4.13 | 4.18 | 4.13 | Clear, colorless, free of visible particle |
| | | | | | | during the entire observation time. |
| 0.12 | 1N—HCl | 4.34 | 4.31 | 4.34 | 4.30 | Clear, colorless, free of visible particle |
| | | | | | | during the entire observation time. |
| | H₃PO₄ | 4.32 | 4.30 | 4.34 | 4.31 | Clear, colorless, free of visible particle |
| | dilute | | | | | during the entire observation time. |
| | Citric acid | 3.98 | 3.89 | 3.93 | 3.90 | Clear, colorless, free of visible particle |
| | | | | | | during the entire observation time. |
| 0.25 | 1N—HCl | 4.28 | 4.25 | 4.28 | 4.26 | Clear, colorless, free of visible particle |
| | | | | | | during the entire observation time. |
| | H₃PO₄ | 4.24 | 4.24 | 4.27 | 4.25 | Clear, colorless, free of visible particle |
| | dilute | | | | | during the entire observation time. |
| | Citric acid | 3.86 | 3.80 | 3.83 | 3.82 | Clear, colorless, free of visible particle |
| | | | | | | during the entire observation time. |

*pH determined directly after mixing the albuterol inhalation solution and the Captisol Budesonide Inhalation Solution

### Example 7

Large scale production of a unit dose formulation comprising an albuterol solution prepared with albuterol free base.

A method for the large scale production approximately 500 mls of a unit dose formulation comprising an albuterol solution prepared with albuterol free base is provided which comprises the following steps: (a) albuterol free base, NaCl, and HCL (1 N) are obtained from a dispensing room; (b) NaCl is added to 80 weight % water; (c) albuterol free base is added to the solution of (b); (d) HCl (1N) is added to the solution of (c); (e) water is added to the solution of (d) in a quantity sufficient to provide a total weight % equal to 100; (f) the solution of (e) is mixed at an appropriate speed for two hours at between 15° C. and 25° C.; (g) the solution of (f) is evaluated under In Process Controls (IPC), which include bioburden, pH, active drug content and osmolality; (h) the solution of (f) is filtered sterilized through a 0.22 micron filter; (i) the filtered solution of (h) is stored in a holding tank at between 5° C. and 25° C. for not longer than 7 days; (j) the solution of (h) is filtered sterilized through a 0.22 micron filter; (k) the filtered solution of (h) is transferred to a buffer tank for filler; (l) the solution of (k) is filtered sterilized through a 0.22 micron filter; (m) the solution of (l) is filled into unit doses consisting of from about 0.52-0.57 mils and held for not longer than 14 days; (n) samples of the unit doses of (m) are evaluated under IPC, which evaluates leakage, fill volume, appearance, the presence of particles, tightness, ability to open the unit dose, and engraving; (o) pouches are prepared containing 5 unit dose vials per pouch; (p) the unit dose vials of (o) are tested prior to release; and (q) the pouches are placed in cartons for shipment.

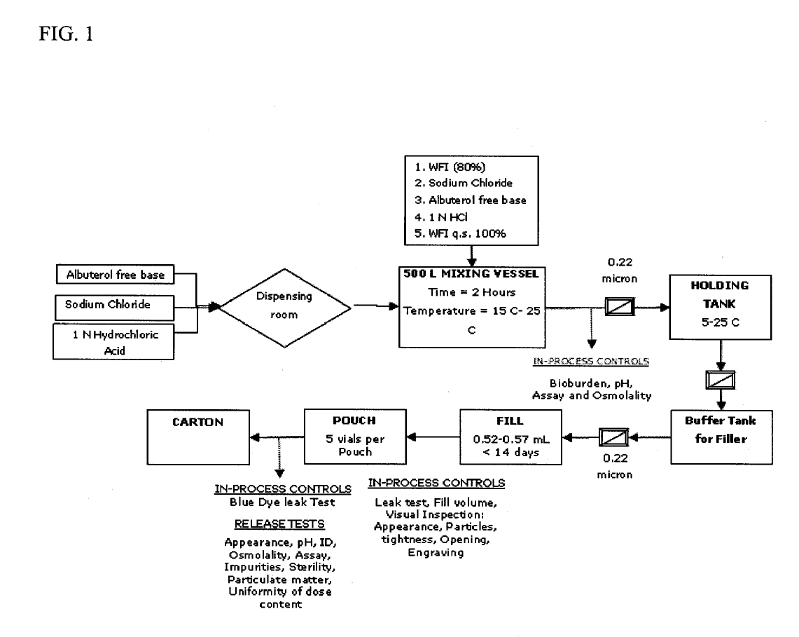

FIG. 1 provides a flowchart setting forth the large scale manufacturing process for the production and packaging of the approximately 0.5 ml unit dose formulations described in Example 7.

### Example 8

A method for the large scale production approximately 500 mls of a unit dose formulation comprising an albuterol solution prepared with albuterol free base is conducted as set for in Example 7, except for the following steps: (a) albuterol free base, NaCl, and citric acid are obtained from a dispensing room; and (d) citric acid is added to the solution of (c).

### Example 9

Three albuterol inhalation solutions are prepared according to the methods set forth below using the following pharmaceutically acceptable agents:

| | Albuterol Concentration [mg/dose] | | |
|---|---|---|---|
| | 0.31 | 0.63 | 1.25 |
| Ingredient | LOW | MEDIUM | HIGH |
| Albuterol free base (weight g) | 0.062 | 0.126 | 0.25 |
| Sodium Chloride NaCl (weight g) | 0.88 g | 0.86 g | 0.84 g |
| Hydrochloric acid (HCl (1N)) | ad ˜pH 3.75 ± 0.15 | ad ˜pH 3.75 ± 0.15 | ad ˜pH 3.75 ± 0.15 |
| Sodium Hydroxide (NaOH) | ad ˜pH 3.75 ± 0.15 | ad ˜pH 3.75 ± 0.15 | ad ˜pH 3.75 ± 0.15 |
| Water (weight mL) | qs to 100 | qs to 100 | qs to 100 |

The albuterol inhalation solutions are formulated according to the following methods: (a) NaCl is added to approximately 80 mLs water to form an aqueous solution; (b) albuterol free base is added to the solution of (a); (c) HCl is added to the solution of (b); and (d) approximately 20 mLs water is added to the solution of (c). The pH is determined using standard chemical techniques known in the art. If the pH of the aqueous inhalation solution comprising (a)-(d) is lower than 3.75±0.15, the process further comprises (e) the addition of NaOH until a pH of 3.75±0.15 is obtained.

### Example 10

Large scale production of a unit dose formulation comprising an albuterol solution prepared with albuterol free base

Three albuterol inhalation solutions were prepared according to the formula set forth below using the following pharmaceutically acceptable agents:

| | 0.31 mg Dose | | 0.63 mg Dose | | 1.25 mg Dose | | | |
|---|---|---|---|---|---|---|---|---|
| | (0.62 mg/mL) | | (1.26 mg/mL) | | (2.50 mg/mL) | | Placebo | |
| | For 1 mL | For 200 L | For 1 mL | For 200 L | For 1 mL | For 200 L | For 1 mL | For 200 L |
| Albuterol | 0.62 mg | 124.0 g | 1.26 mg | 252.0 g | 2.5 mg | 500.0 g | | |
| Sodium Chloride | 8.8 mg | 1760 g | 8.6 mg | 1720 g | 8.4 mg | 1680 g | 8.9 mg | 1800 g |
| 1 N Hydrochloric | q.s. to pH | q.s. to pH | q.s. to pH | q.s. to pH | q.s. to pH | q.s. to pH | q.s. to pH | q.s. to pH |
| acid (volumes are | 3.75 ± 0.15 | 3.75 ± 0.15 | 3.75 ± 0.15 | 3.75 ± 0.15 | 3.75 ± 0.15 | 3.75 ± 0.15 | 3.75 ± 0.15 | 3.75 ± 0.15 |
| approximates) | (2.6 mL) | (520 mL) | (5.2 mL) | (1040 mL) | (10.69 mL) | (2137 mL) | | |
| 1 N Sodium | q.s. to pH | q.s. to pH | q.s. to pH | q.s. to pH | q.s. to pH | q.s. to pH | q.s. to pH | q.s. to pH |
| Hydroxide | 3.75 ± 0.15 | 3.75 ± 0.15 | 3.75 ± 0.15 | 3.75 ± 0.15 | 3.75 ± 0.15 | 3.75 ± 0.15 | 3.75 ± 0.15 | 3.75 ± 0.15 |
| Water for Injection | q.s. 1 mL | q.s. 200 kg | q.s. 1 mL | q.s. 200 kg | q.s. 1 mL | q.s. 200 kg | q.s. 1 mL | q.s. 200 kg |

The actual amounts for the pharmaceutically acceptable agents used in the large scale production unit dose formulation comprising an albuterol solution were:

| Strength | AIS 0.31 mg | | AIS 0.63 mg | | AIS 1.25 mg | | Placebo | |
|---|---|---|---|---|---|---|---|---|
| Albuterol | 124.0 | g | 252.0 | g | 500.0 | g | — | |
| Sodium | 1760.0 | g | 1720.0 | g | 1680.0 | g | 1800 | g |
| Chloride | | | | | | | | |
| 1 N | 551.65 | mL | 1080.68 | mL | 2211.6 | mL | 384 | mL |
| Hydrochloric | | | | | | | | |
| acid | | | | | | | | |
| 1 N Sodium | — | | — | | 92.1 | mL | — | |
| Hydroxide | | | | | | | | |
| Water for | q.s. 200 | kg | q.s. 200 | kg | q.s. 200 | kg | q.s. 200 | kg |
| Injection | | | | | | | | |

A method for the large scale production approximately 200 L of a unit dose formulation comprising an albuterol solution prepared with albuterol free base is provided which comprises the following steps: (a) Check the production area for environmental cleanliness; (b) Sanitize the formulation tank with sterilized steam; (c) Add about 120% (220 kg) of WFI required to prepare the batch and cool the water to 25-30° C.; (d) Activate the mixer. Remove the cooled WFI from the tank until approximately 80% (160 Kg) of the final batch weight remains; (e) Slowly add required amount of Sodium Chloride to the formulation tank. Mix until visually clear; (f) Add required amount of Albuterol free base to the formulation tank. Mix until visually clear: (g) Add 1.0 N Hydrochloric acid as indicated by the above-described formula

Take sample for pH

Add additional 1.0 N Hydrochloric acid or 1.0 N Sodium Hydroxide to adjust the pH to 3.75±0.15. Mix for a minimum of 10 minutes after each addition before taking the sample for measuring pH.

Repeat pH sampling and acid/base adjustment until pH target is met;

(h) Turn off the mixer and let the batch stabilize until load cell readings are constant. Slowly add previously cooled (25-30° C.) WFI to the formulation tank until the final batch weight is achieved; (i) Turn on the mixer and mix the solution for minimum of 10 minutes; (j) Take appropriate number of samples from the bulk solution for chemistry (pH, osmolality, assay, oxygen content) and biological testing (bioburden and microbial validation, if necessary); (k) If assay, pH and osmolality tests are within specifications, release the batch for further processing; (l) Filter the bulk formulation aseptically in a previously sterilized holding tank. 0.1 micron Pall Ultipore nylon filter was used for product filtration; (m) Fill approximately 0.5 mL of the product aseptically into sterile vials produced in a previously sterilized blow-fill-seal machine. Two 0.1 micron Pall Ultipore nylon filters are used for transferring the product from holding tank into the vials; and (n) Place 4 vials in Aluminum foil overwrap. Print lot number on the overwrap.

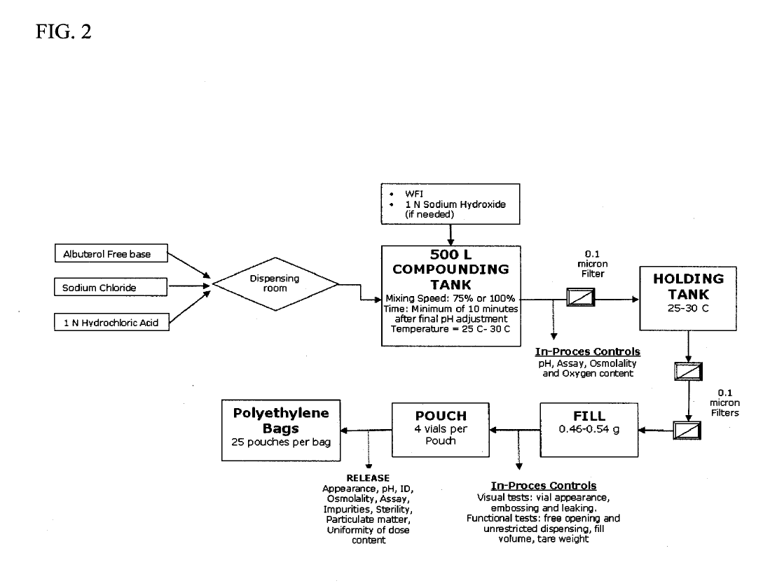

FIG. 2 provides a flowchart setting forth the large scale manufacturing process for the production and packaging of the approximately 0.5 ml unit dose formulations described in the present Example.

What is claimed is:

1. A unit dose formulation comprising a therapeutically effective amount of an albuterol solution prepared by the process comprising the steps of:
a. providing about 0.84 to about 0.90 weight % NaCl;
b. mixing said NaCl with about 80 weight % water to form an aqueous solution;
c. adding from about 0.03 to about 0.25 weight % albuterol free base to the solution of (b);
d. adding about 0.10 to about 1.3 weight % HCl (1N) to the solution of (c);
e. optionally adding about 0.001 to about 0.05 weight % NaOH to the solution of (d);
f. adding water to the solution of (d) in a quantity sufficient to provide a total weight % equal to 100;
g. filter sterilizing the solution of (e); and
h. dividing the solution of (f) into about 0.5 ml unit doses;
wherein said unit dose formulation has a molar ratio of starting albuterol free base: HCl of about 0.85 to about 1.20, a pH of about 3.0 to about 4.5, and wherein said unit dose formulation comprises a therapeutically effective amount of albuterol and is suitable for inhalation therapy via nebulization.

2. The unit dose formulation of claim 1, wherein said unit dose formulation is prepared by the process comprising the steps of:
a. providing about 0.90 weight % NaCl;
b. mixing said NaCl with about 80 weight % water to form an aqueous solution;
c. adding about 0.03 weight % albuterol free base to the solution of (b);
d. adding about 0.17 weight % HCl (1N) to the solution of (c);
e. optionally adding about 0.001 to about 0.05 weight % NaOH to the solution of (d);
f. adding water to the solution of (d) in a quantity sufficient to provide a total weight % equal to 100;
g. filter sterilizing the solution of (e); and
h. dividing the solution of (f) into about 0.5 ml unit doses;
wherein said unit dose formulation has a molar ratio of starting albuterol free base: HCl of about 0.87, a pH of about 3.7, and wherein said unit dose formulation comprises a therapeutically effective amount of albuterol and is suitable for inhalation therapy via nebulization.

3. The unit dose formulation of claim 1, wherein said unit dose formulation is prepared by the process comprising the steps of:
a. providing about 0.87 weight % NaCl;
b. mixing said NaCl with about 80 weight % water to form an aqueous solution;
c. adding about 0.12 weight % albuterol free base to the solution of (b);
d. adding about 0.55 weight % HCl (1N) to the solution of (c);
e. optionally adding about 0.001 to about 0.05 weight % NaOH to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: HCl is about 1.08, a pH of about 3.6, and wherein said unit dose formulation comprises a therapeutically effective amount of albuterol and is suitable for inhalation therapy via nebulization.

4. The unit dose formulation of claim 1, wherein said unit dose formulation is prepared by the process comprising the steps of:
a. providing about 0.88 weight % NaCl;
b. mixing said NaCl with about 80 weight % water to form an aqueous solution;
c. adding about 0.062 weight % albuterol free base to the solution of (b);
d. adding about 0.33 weight % HCl (1N) to the solution of (c);
e. optionally adding about 0.001 to about 0.05 weight % NaOH to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: HCl is about 0.94, a pH of about 3.75±0.15, and wherein said unit dose formulation comprises a therapeutically effective amount of albuterol and is suitable for inhalation therapy via nebulization.

5. The unit dose formulation of claim 1, wherein said unit dose formulation is prepared by the process comprising the steps of:
a. providing about 0.86 weight % NaCl;
b. mixing said NaCl with about 80 weight % water to form an aqueous solution;
c. adding about 0.126 weight % albuterol free base to the solution of (b);
d. adding about 0.64 weight % HCl (1N) to the solution of (c);
e. optionally adding about 0.001 to about 0.05 weight % NaOH to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: HCl is about 0.97, a pH of about 3.75±0.15, and wherein said unit dose formulation comprises a therapeutically effective amount of albuterol and is suitable for inhalation therapy via nebulization.

6. The unit dose formulation of claim 1, wherein said unit dose formulation is prepared by the process comprising the steps of:
a. providing about 0.84 weight % NaCl;
b. mixing said NaCl with about 80 weight % water to form an aqueous solution;
c. adding about 0.25 weight % albuterol free base to the solution of (b);
d. adding about 1.30 weight % HCl (1N) to the solution of (c);
e. optionally adding about 0.001 to about 0.05 weight % NaOH to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: HCl is about 0.95, a pH of about 3.75±0.15, and wherein said unit dose formulation comprises a therapeutically effective amount of albuterol and is suitable for inhalation therapy via nebulization.

7. The unit dose formulation of claim 1, wherein said unit dose formulation is prepared by the process comprising the steps of:
a. providing about 0.84 weight % NaCl;
b. mixing said NaCl with about 80 weight % water to form an aqueous solution;
c. adding about 0.25 weight % albuterol free base to the solution of (b);
d. adding about 1.06 weight % HCl (1N) to the solution of (c);
e. optionally adding about 0.001 to about 0.05 weight % NaOH to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: HCl of about 1.16, a pH of about 3.4, and wherein said unit dose formulation comprises a therapeutically effective amount of albuterol and is suitable for inhalation therapy via nebulization.

8. A unit dose formulation comprising a therapeutically effective amount of an albuterol solution prepared by the process comprising the steps of:
a. providing about 0.84 to about 0.90 weight % NaCl;
b. mixing said NaCl with about 80 weight % water to form an aqueous solution;
c. adding from about 0.03 to about 0.25 weight % albuterol free base to the solution of (b);
d. adding about 0.03 to about 0.35 weight % citric acid to the solution of (c);
e. optionally adding about 0.001 to about 0.05 weight % NaOH to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: citric acid of about 0.80 to about 1.0, a pH of about 3.0 to about 4.5, and wherein said unit dose formulation comprises a therapeutically effective amount of albuterol and is suitable for inhalation therapy via nebulization.

9. The unit dose formulation of claim 8, wherein said unit dose formulation is prepared by the process comprising the steps of:
a. providing about 0.90 weight % NaCl;
b. mixing said NaCl with about 80 weight % water to form an aqueous solution;
c. adding about 0.03 weight % albuterol free base to the solution of (b);
d. adding about 0.03 weight % citric acid to the solution of (c);
e. optionally adding about 0.001 to about 0.05 weight % sodium citrate to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: citric acid is about 0.80, a pH of about 3.7, and wherein said unit dose formulation comprises a therapeutically effective amount of albuterol and is suitable for inhalation therapy via nebulization.

10. The unit dose formulation of claim 8, wherein said unit dose formulation is prepared by the process comprising the steps of:
a. providing about 0.87 weight % NaCl;
b. mixing said NaCl with about 80 weight % water to form an aqueous solution;
c. adding about 0.12 weight % albuterol free base to the solution of (b);
d. adding about 0.11 weight % citric acid to the solution of (c);
e. optionally adding about 0.001 to about 0.05 weight % sodium citrate to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: citric acid of about 0.88, a pH of about 3.7, and wherein said unit dose formulation comprises a therapeutically effective amount of albuterol and is suitable for inhalation therapy via nebulization.

11. The unit dose formulation of claim 8, wherein said unit dose formulation is prepared by the process comprising the steps of:
a. providing about 0.84 weight % NaCl;
b. mixing said NaCl with about 80 weight % water to form an aqueous solution;
c. adding about 0.25 weight % albuterol free base to the solution of (b);
d. adding about 0.21 weight % citric acid to the solution of (c);
e. optionally adding about 0.001 to about 0.05 weight % sodium citrate to the solution of (d);
f. adding water to the solution of (e) in a quantity sufficient to provide a total weight % equal to 100;
g. filter sterilizing the solution of (e); and
h. dividing the solution of (g) into about 0.5 ml unit doses;
wherein said unit dose formulation has a molar ratio of starting albuterol free base: citric acid of about 0.96, a pH of about 3.7, and wherein said unit dose formulation comprises a therapeutically effective amount of albuterol and is suitable for inhalation therapy via nebulization.

12. A unit dose formulation comprising a therapeutically effective amount of an albuterol solution prepared by the process comprising the steps of:
a. providing about 0.84 to about 0.90 weight % NaCl;
b. mixing said NaCl with about 80 weight % water to form an aqueous solution;
c. adding from about 0.03 to about 0.25 weight % albuterol free base to the solution of (b);
d. adding about 0.10 to about 1.20 weight % H₃PO₄ to the solution of (c);
e. optionally adding about 0.001 to about 0.05 weight % sodium phosphate dibasic to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: H₃PO₄ of about 0.80 to about 1.20, a pH of about 3.0 to about 4.5, and wherein said unit dose formulation comprises a therapeutically effective amount of albuterol and is suitable for inhalation therapy via nebulization.

13. The unit dose formulation of claim 12, wherein said unit dose formulation is prepared by the process comprising the steps of:
a. providing about 0.90 weight % NaCl;
b. mixing said NaCl with about 80 weight % water to form an aqueous solution;
c. adding about 0.03 weight % albuterol free base to the solution of (b);
d. adding about 0.10 weight % H₃PO₄ to the solution of (c);
e. optionally adding about 0.001 to about 0.05 weight % sodium phosphate dibasic to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: H₃PO₄ of about 0.80 to about 1.20, a pH of about 3.7, and wherein said unit dose formulation comprises a therapeutically effective amount of albuterol and is suitable for inhalation therapy via nebulization.

14. The unit dose formulation of claim 12, wherein said unit dose formulation is prepared by the process comprising the steps of:
a. providing about 0.87 weight % NaCl;
b. mixing said NaCl with about 80 weight % water to form an aqueous solution;
c. adding about 0.12 weight % albuterol free base to the solution of (b);
d. adding about 0.34 weight % H₃PO₄ to the solution of (c);
e. optionally adding about 0.001 to about 0.05 weight % sodium phosphate dibasic to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: H₃PO₄ of about 0.80 to about 1.20, a pH of about 3.7, and wherein said unit dose formulation comprises a therapeutically effective amount of albuterol and is suitable for inhalation therapy via nebulization.

15. The unit dose formulation of claim 12, wherein said unit dose formulation is prepared by the process comprising the steps of:
a. providing about 0.84 weight % NaCl;
b. mixing said NaCl with about 80 weight % water to form an aqueous solution;
c. adding about 0.25 weight % albuterol free base to the solution of (b);
d. adding about 1.02 weight % H₃PO₄ to the solution of (c);
e. optionally adding about 0.001 to about 0.05 weight % sodium phosphate dibasic to the solution of (d);
f. adding water to the solution of (e) in a quantity sufficient to provide a total weight % equal to 100;
g. filter sterilizing the solution of (f); and
h. dividing the solution of (g) into about 0.5 ml unit doses;
wherein said unit dose formulation has a molar ratio of starting albuterol free base: H₃PO₄ of about 0.80 to about 1.20, a pH of about 3.7, and wherein said unit dose formulation comprises a therapeutically effective amount of albuterol and is suitable for inhalation therapy via nebulization.

16. A unit dose formulation comprising a therapeutically effective amount of an albuterol solution prepared by the process comprising the steps of:
a. providing about 0.84 to about 0.90 weight % NaCl;
b. mixing said NaCl with about 80 weight % water to form an aqueous solution;
c. adding from about 0.03 to about 0.25 weight % albuterol free base to the solution of (b);
d. adding about 0.001 to about 5.0 weight % of a stabilizing agent to the solution of (c);
e. adding about 0.10 to about 1.3 weight % HCl (1N) to the solution of (d);
f. optionally adding about 0.001 to about 0.05 weight % NaOH to the solution of (e);
g. adding water to the solution of (f) in a quantity sufficient to provide a total weight % equal to 100;
h. filter sterilizing the solution of (g); and
i. dividing the solution of (h) into about 0.5 ml unit doses;
wherein said unit dose formulation has a molar ratio of starting albuterol free base: HCl of about 0.85 to about 1.20, a pH of about 3.0 to about 4.5, and wherein said unit dose formulation comprises a therapeutically effective amount of albuterol and is suitable for inhalation therapy via nebulization.

17. A unit dose formulation comprising a therapeutically effective amount of an albuterol solution prepared by the process comprising the steps of:
a. providing about 0.84 to about 0.90 weight % NaCl;
b. mixing said NaCl with about 80 weight % water to form an aqueous solution;
c. adding from about 0.03 to about 0.25 weight % albuterol free base to the solution of (b);
d. adding about 0.001 to about 5.0 weight % of a stabilizing agent to the solution of (c);
e. adding about 0.03 to about 0.35 weight % citric acid to the solution of (d);
f. optionally adding about 0.001 to about 0.05 weight % sodium citrate to the solution of (e);
g. adding water to the solution of (f) in a quantity sufficient to provide a total weight % equal to 100;
h. filter sterilizing the solution of (g); and
i. dividing the solution of (h) into about 0.5 ml unit doses;
wherein said unit dose formulation has a molar ratio of starting albuterol free base: citric acid of about 0.80 to about 1.0, a pH of about 3.0 to about 4.5, and wherein said unit dose formulation comprises a therapeutically effective amount of albuterol and is suitable for inhalation therapy via nebulization.

18. A unit dose formulation comprising a therapeutically effective amount of an albuterol solution prepared by the process comprising the steps of:
a. providing about 0.84 to about 0.90 weight % NaCl;
b. mixing said NaCl with about 80 weight % water to form an aqueous solution;
c. adding from about 0.03 to about 0.25 weight % albuterol free base to the solution of (b);
d. adding about 0.001 to about 5.0 weight % of a stabilizing agent to the solution of (c);
e. adding about 0.10 to about 1.20 weight % H₃PO₄ to the solution of (d);
f. optionally adding about 0.001 to about 0.05 weight % sodium phosphate dibasic to the solution of (e);
g. adding water to the solution of (f) in a quantity sufficient to provide a total weight % equal to 100;
h. filter sterilizing the solution of (g); and
i. dividing the solution of (h) into about 0.5 ml unit doses;
wherein said unit dose formulation has a molar ratio of starting albuterol free base: H₃PO₄ of about 0.80 to about 1.20, a pH of about 3.0 to about 4.5, and wherein said unit dose formulation comprises a therapeutically effective amount of albuterol and is suitable for inhalation therapy via nebulization.

19. The unit dose formulation of any of claims 16-18, wherein said stabilizing agent is selected from the group consisting of a chelating agent, a preservative, an antioxidant or a combination thereof.

20. The unit dose formulation for claim 19, wherein said stabilizing agent is a chelating agent.

21. The unit dose formulation of claim 20, wherein said chelating agent is EDTA.

22. The unit dose formulation for claim 19, wherein said stabilizing agent is a preservative.

23. The unit dose formulation of claim 19, wherein said preservative is benzalkonium chloride.

24. The unit dose formulation for claim 19, wherein said stabilizing agent is an antioxidant.

25. The unit dose formulation of claim 24, wherein said antioxidant is selected from the group consisting of sodium ascorbate, sodium citrate, alpha tocopherol, vitamin E, or combinations thereof.

26. The unit dose formulation of any one of claims 1-25, further comprising a second pharmaceutically active agent.

27. The unit dose formulation of claim 26, wherein said second pharmaceutically active agent is a corticosteroid.

28. The unit dose formulation of claim 26, wherein said second pharmaceutically active agent is an antibiotic.

29. The unit dose formulation of claim 26, wherein said second pharmaceutically active agent is an anti-cholinergic agent.

30. The unit dose formulation of claim 26, wherein said second pharmaceutically active agent is a dopamine (D₂) receptor agonist.

31. A method of treating a patient diagnosed with, or suspected of having, a bronchoconstrictive disorder, comprising the administration of the unit dose formulation of any one of claims 1-30.

32. The method of claim 32, wherein the bronchoconstrictive disorder is selected from the group consisting of asthma, pediatric asthma, bronchial asthma, allergic asthma, occupational asthma, aspirin sensitive asthma, exercise-induced asthma, intrinsic asthma, chronic obstructive pulmonary disease (COPD), chronic bronchitis, cystic fibrosis and emphysema.
